(19) United States
(12) Patent Application Publication
(10) Pub. No.: US 2018/0235791 A1
(43) Pub. Date: Aug. 23, 2018

(54) INSERTING DEVICE OF STENT

(71) Applicant: S&G BIOTECH, INC., Seongnam-si (KR)

(72) Inventors: Sung Kwon Kang, Yongin-si (KR); Sun Soon Park, Seongnam-si (KR); So Hee Jung, Gwangju-si (KR)

(73) Assignee: S&G BIOTECH, INC., Seongnam-si (KR)

(21) Appl. No.: 15/903,050

(22) Filed: Feb. 23, 2018

(30) Foreign Application Priority Data
Feb. 23, 2017 (KR) 10-2017-0024067

(51) Int. Cl. A61F 2/954; A61F 2/962

(52) U.S. Cl. CPC A61F 2/954; A61F 2002/9505; A61F 2/962

(57) ABSTRACT

Disclosed is a stent insertion apparatus including a sheath formed to have a hollow shape and in which a stent is accommodated, a main guide tube movably inserted into the sheath and to which one end of the stent is coupled, and an auxiliary guide tube coupled to the main guide tube in a bifurcated manner and to which the other end of the stent, which branches from the one end of the stent, is coupled.

### CROSS-REFERENCE TO RELATED APPLICATION

This application claims priority under 35 U.S.C. § 119 from Korean Patent Application No. 10-2017-0024067, filed on Feb. 23, 2017, the disclosure of which is incorporated herein by reference in its entirety.

### BACKGROUND

### Field of the Invention

The present invention relates to a stent insertion apparatus, and more particularly, to a stent insertion apparatus for inserting a bifurcating stent into a human body.

### Discussion of Related Art

Generally, when stenosis occurs due to a cause such as a deposit and like in organs such as a blood vessel, an esophagus, a bile duct, and like, which need to secure a certain inner diameter, a stent may be inserted to artificially widen a stenosed part.

Particularly, since a bile duct is used as a movement path for bile which is a digestive enzyme generated by a liver, when stenosis occurs, a diameter of the bile duct decreases such that bile may not be smoothly discharged.

Accordingly, a stent is inserted into the stenosed bile duct and widens the bile duct to smoothly discharge the bile such that reflux of food or fluids may be prevented.

However, the bile duct bifurcates and forms a Y shape such that it is difficult to install a stent in a bifurcating part of the bile duct.

For example, in Korean Patent Publication No. 10-2012-0035978 (Related Art Document), a stent is installed in a first bifurcating tube and then a stent is installed in a second bifurcating tube. Merely, the stent installed in the first bifurcating tube and the stent installed in the second bifurcating tube overlap each other to form a Y shape.

Since it is necessary to insert a stent insertion apparatus into a human body two times to install the stents in the first bifurcating tube and the second bifurcating tube like the Related Art Document, the entire installation time may increase.

Also, bile may not smoothly flow through a part at which the two stents overlap each other.

### RELATED ART DOCUMENT

### Patent Document

Korean Patent Publication No. 10-2012-0035978 (published on Apr. 17, 2012)

### SUMMARY OF THE INVENTION

One aspect of the present invention provides a stent insertion apparatus for inserting a bifurcating stent into a bifurcating part of a bile duct.

Aspects of the present invention are not limited to the above aspect, and other unstated aspects will be clearly understood by one of ordinary skill in the art from the following description.

According to one aspect of the present invention, a stent insertion apparatus includes a sheath formed to have a hollow shape and in which a stent is accommodated, a main guide tube movably inserted into the sheath and to which one end of the stent is coupled, and an auxiliary guide tube coupled to the main guide tube in a bifurcated manner and to which the other end of the stent, which branches from the one end of the stent, is coupled.

The stent insertion apparatus may include a first guide wire movably inserted into the main guide tube to guide movement of the main guide tube and a second guide wire inserted into the sheath through a through hole formed in an outer surface of the sheath and inserted into the auxiliary guide tube to guide movement of the auxiliary guide tube.

The stent insertion apparatus may include olive tips respectively provided at front ends of the main guide tube and the auxiliary guide tube and formed to have a hollow shape to allow the first guide wire and the second guide wire to be inserted thereinto.

The stent insertion apparatus may further include a support member into which the main guide tube is inserted, to which the auxiliary guide tube is coupled at an outside thereof, and which is provided at a rear end of the stent to prevent the stent from moving.

A marker may be provided at a front end of the support member to allow a position of the stent to be confirmed.

The stent insertion apparatus may include a holding member formed to protrude from each of circumferences of the main guide tube and the auxiliary guide tube to prevent the stent from moving.

The stent insertion apparatus may include fixing members which fix the one end and other end of the stent to the main guide tube and the auxiliary guide tube, respectively, to prevent the stent from extending.

The stent insertion apparatus may include a guide tube handle provided at one end of the main guide tube to communicate with the main guide tube and a sheath handle coupled to and communicate with the sheath to allow the main guide tube to be inserted thereinto.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1 and 2 are views illustrating an appearance of a stent insertion apparatus according to one embodiment of the present invention;

FIG. 3 is a perspective view of the stent insertion apparatus according to one embodiment of the present invention;

FIG. 4 is a cross-sectional view of the stent insertion apparatus according to one embodiment of the present invention;

FIG. 5 is a view illustrating an appearance of a stent installed in a biliary tract using the stent insertion apparatus according to one embodiment of the present invention; and

FIGS. 6 to 10 are views illustrating a process of installing a stent using the stent insertion apparatus according to one embodiment of the present invention.

### DETAILED DESCRIPTION OF EXEMPLARY EMBODIMENTS

Hereinafter, one embodiment of the present invention will be described in detail with reference to the attached drawings. However, it will be easily understood by one of ordinary skill in the art that the attached drawings are provided to more easily disclose the content of the present invention and the scope of the present invention is not limited to the scope of the attached drawings.

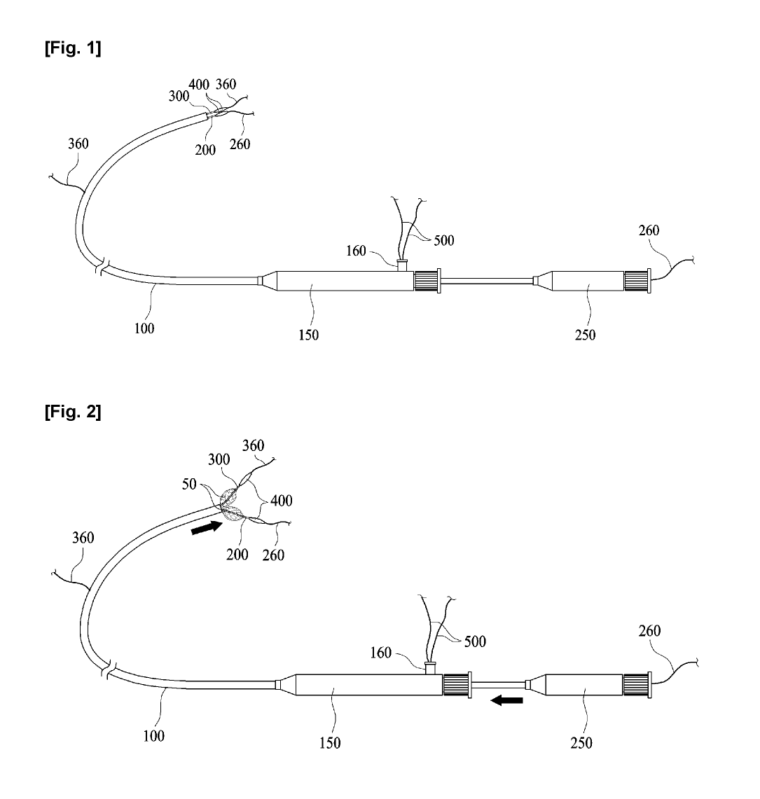

Referring to FIGS. 1 to 4, a stent insertion apparatus according to one embodiment of the present invention includes a sheath 100, a main guide tube 200, and an auxiliary guide tube 300.

The sheath 100 has a hollow shape and may accommodate a stent 50 therein.

The sheath 100 may have a hollow shape to form an accommodation space therein and may accommodate the main guide tube 200, the auxiliary guide tube 300, and the like therein in addition to the stent 50.

The sheath 100 may be used for transferring the stent 50, the main guide tube 200, and the auxiliary guide tube 300, which are accommodated therein, to an installation position of the stent 50. Accordingly, the sheath 100 may be formed to be flexible to be insertable into a human body and may be formed of a material generally used for a medical apparatus.

Meanwhile, the stent 50 accommodated in the sheath 100 is formed to be expandable and contractable, is accommodated in the sheath 100 in a contracted state, is moved toward the installation position, is discharged from the sheath 100, and is expanded such that a stenosed part may be expanded.

Next, the main guide tube 200 and the auxiliary guide tube 300 accommodated in the sheath 100 will be described.

The main guide tube 200 may be movably inserted into the sheath 100, and one end of the stent 50 may be coupled thereto.

The auxiliary guide tube 300 may be coupled to the main guide tube 200 in a bifurcated manner, and the other end branching from the one end of the stent 50 may be coupled thereto.

The one end and other end of the stent 50 may be fixed to the main guide tube 200 and the auxiliary guide tube 300 by a fixing member 500.

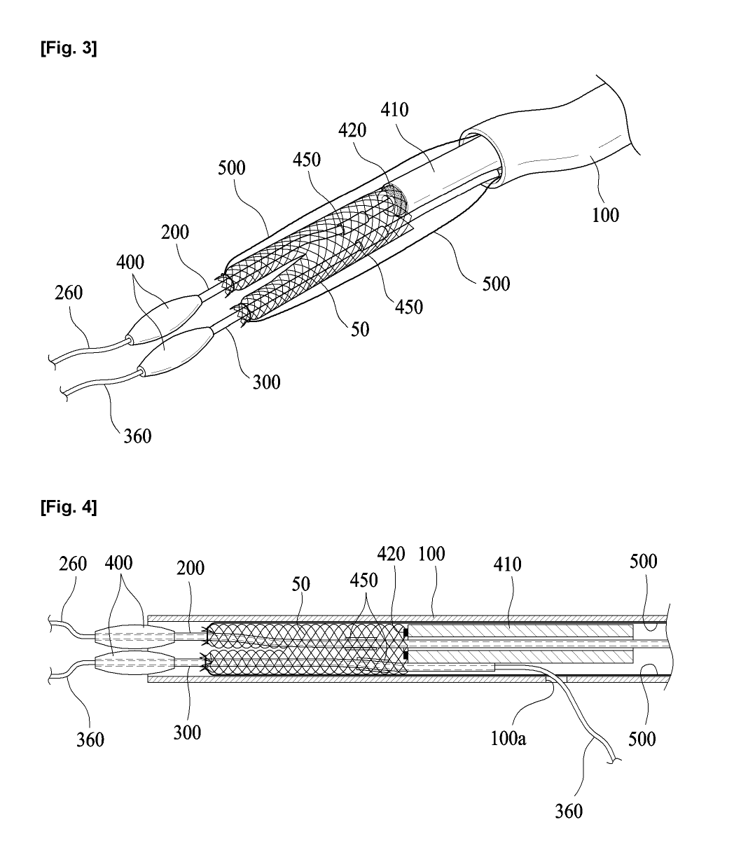

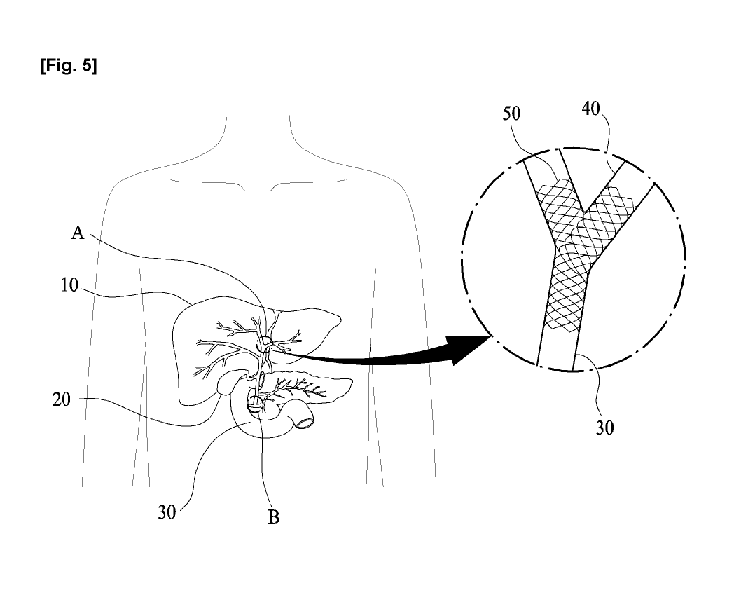

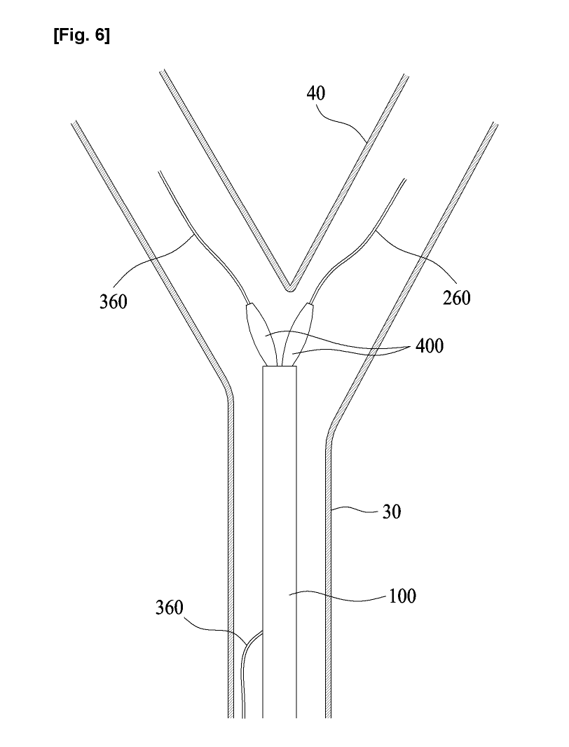

As shown in FIGS. 3 and 4, the fixing member 500 may be formed to have a wire shape to deploy the stent 50 while being pulled.

However, the fixing member 500 is not limited to the wire shape and may be formed to have, for example, a hook shape and may fix the one end and other end of the stent 50 so as not to be unfolded.

The fixing member 500 may be manufactured to have an elastic force to be separable from the stent 50 due to extendibility of the stent 50 when the stent 50 is discharged outward from the sheath 100 or may be formed of a biodegradable material so as to be degraded in a human body after a certain period of time.

Hereinafter, for convenience for description, a case in which the fixing member 500 is used as a fixing wire 500 which has a wire shape will be described.

Meanwhile, the stent 50 to be installed by the stent insertion apparatus according to one embodiment of the present invention may have a bifurcating shape as shown in FIG. 5 and may be installed at each of installation positions A and B at which bifurcating bile ducts 40 bifurcate from a bile duct 30.

The stent insertion apparatus according to one embodiment of the present invention may install the stent 50 having the bifurcating shape at an installation position through a single process.

In detail, the main guide tube 200 and the auxiliary guide tube 300 may be simultaneously inserted into an integrated rear end of the stent 50. Here, the main guide tube 200 may be inserted into and coupled to one end branching from the rear end of the stent 50 and the auxiliary guide tube 300 may be inserted into and coupled to the other end branching from the rear end of the stent 50.

Although the main guide tube 200 and the auxiliary guide tube 300 are included to install the stent 50 having the bifurcating shape, it is necessary to decrease a diameter of the sheath 100 to be easily inserted into a human body.

Accordingly, in the stent insertion apparatus according to one embodiment of the present invention, the auxiliary guide tube 300 is provided at only a front end of the sheath 100 and a second guide wire 360 which will be described below is inserted into the sheath 100 through an outer surface of the sheath 100 to decrease the diameter of the sheath 100.

In detail, the main guide tube 200 may be formed to be longer than the sheath 100 and may be inserted into the sheath 100 through a sheath handle 150 provided at one end of the sheath 100.

The sheath handle 150 may be coupled to and communicate with the sheath 100 to insert the main guide tube 200. A protrusion portion 160 may protrude from an outer surface of the sheath handle 150 to insert the above-described fixing wire 500.

Also, a guide tube handle 250 may be provided at one end of the main guide tube 200 to communicate with the main guide tube 200.

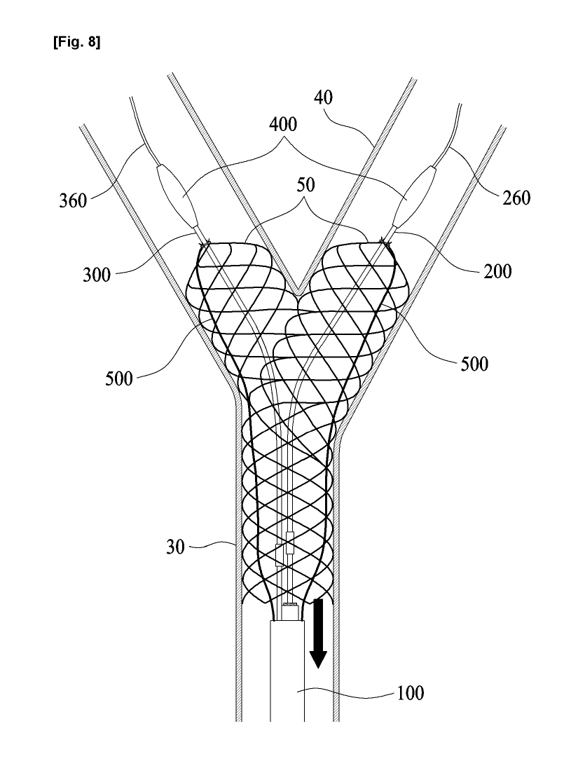

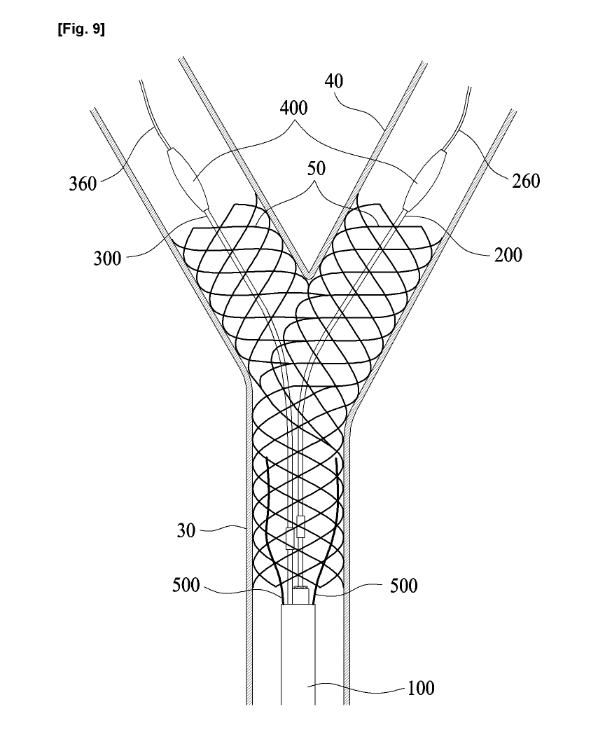

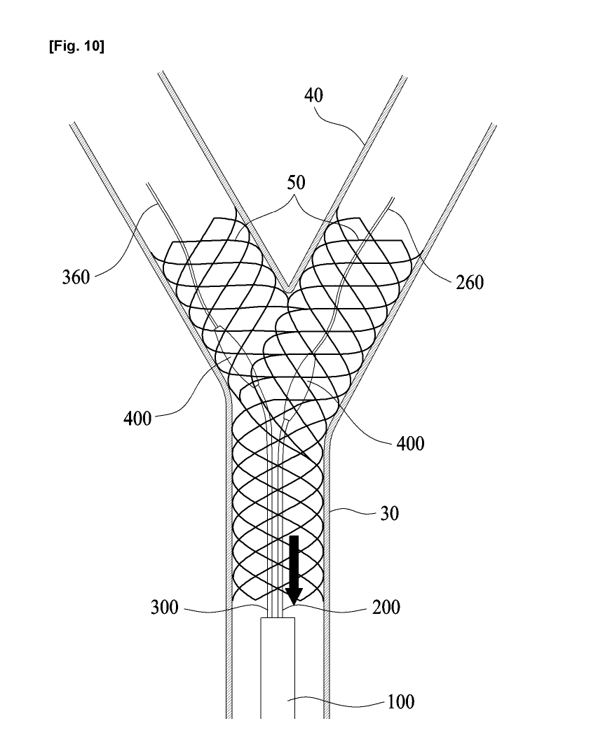

Accordingly, the sheath handle 150 and the guide tube handle 250 are relatively moved such that the main guide tube 200 and the sheath 100 may relatively move. A process of installing the stent 50 according to the relative movement of the main guide tube 200 and the sheath 100 will be described with reference to FIGS. 6 to 10.

A first guide wire 260, which is movably inserted into the main guide tube 200 and guides the movement of the main guide tube 200, may be inserted into a hollow of the guide tube handle 250.

The main guide tube 200 may move as the guide tube handle 250 is adjusted, and the auxiliary guide tube 300 may move as the main guide tube 200 moves.

Also, the first guide wire 260 may be discharged from the main guide tube 200 to the outside of the sheath 100, may detect a movement direction of the main guide tube 200, and may guide the movement of the main guide tube 200.

Like the main guide tube 200, the second guide wire 360, which guides the movement of the auxiliary guide tube 300, may be inserted into the auxiliary guide tube 300.

That is, the second guide wire 360 may be inserted into the sheath 100 through a through hole 100a formed at the outer surface of the sheath 100 and may be inserted into the auxiliary guide tube 300 to guide movement of the auxiliary guide tube 300.

Here, the first guide wire 260 is inserted into the main guide tube 200 through the guide tube handle 250, while the second guide wire 360 may be inserted into the sheath 100 through the outer surface of the sheath 100.

As described above, in the embodiment of the present invention, since the auxiliary guide tube 300 is provided at the front end of the sheath 100 to decrease the diameter of the sheath 100, a length of the auxiliary guide tube 300 may be shorter than that of the main guide tube 200.

Accordingly, the second guide wire 360 may be inserted into the sheath 100 through the through hole 100a formed in the outer surface of the sheath 100 instead of being inserted into the sheath 100 through the guide tube handle 250.

Also, a position at which the through hole 100a is formed may be located at the rear of a position at which the auxiliary guide tube 300 is inserted into the sheath 100 such that the second guide wire 360 may be smoothly inserted into the sheath 100 and the auxiliary guide tube 300.

As described above, the stent insertion apparatus according to one embodiment of the present invention may include the main guide tube 200 and the auxiliary guide tube 300 to install the stent 50 having a bifurcating shape and the diameter of the sheath 100 may be reduced by forming the auxiliary guide tube 300 having a shorter length than that of the main guide tube 200 such that the sheath 100 may be easily inserted into a human body.

Meanwhile, olive tips 400 may be provided at front ends of the main guide tube 200 and the auxiliary guide tube 300.

The olive tips 400 may be respectively provided at the front ends of the main guide tube 200 and the auxiliary guide tube 300 and may have a hollow shape such that the first guide wire 260 and the second guide wire 360 are inserted thereinto.

The olive tips 400 may move as the main guide tube 200 moves and may be discharged outward from the sheath 100 to guide the movements of the main guide tube 200 and the auxiliary guide tube 300 with the first guide wire 260 and the second guide wire 360.

Also, the olive tip 400 may be formed to have a curved surface to be easily inserted into a human body and may be formed to gradually acuminate from a center toward a front end and a rear end.

Meanwhile, referring to FIGS. 3 and 4, a support member 410, a marker 420, a holding member 450, a fixing wire 500, and the like may be further included in the sheath 100.

The main guide tube 200 may be inserted into the support member 410, the auxiliary guide tube 300 may be coupled to the outside of the support member 410, and the support member 410 may be provided at the rear end of the stent 50 to prevent the stent 50 from moving.

That is, the support member 410 may extend the main guide tube 200 in a radial direction and may support the rear end of the stent 50 to prevent the stent 50 from moving according to relative movement of the main guide tube 200 or the sheath 100.

Referring to FIGS. 3 and 4, the auxiliary guide tube 300 may be coupled to the main guide tube 200 through the support member 410 but is not limited thereto.

For example, the auxiliary guide tube 300 may be directly coupled to the main guide tube 200 and the support member 410 may be provided at a rear end of the auxiliary guide tube 300. Here, the support member 410 may be formed to have a greater diameter than that of the stent 50 and may support the stent 50 to prevent the stent 50 from moving.

Also, the marker 420 may be provided at a front end of the support member 410 such that a position of the stent 50 is confirmed.

The marker 420 is for confirming the position of the stent 50 from the outside during a process in which the stent 50 is inserted and may be formed of, for example, a material capable of being shown by X-rays.

Accordingly, the position of the stent 50 may be confirmed through the marker 420 and the stent 50 may be precisely installed at an installation position.

Meanwhile, although the marker 420 is provided at the front end of the support member 410 and displays the rear end of the stent 50, the marker 420 may be formed at the main guide tube 200 or the auxiliary guide tube 300 as well as the rear end of the stent 50 and may display the branching one end or other end of the stent 50.

Meanwhile, the holding member 450 may be formed to protrude from each of circumferences of the main guide tube 200 and the auxiliary guide tube 300 to prevent the stent 50 from moving.

The holding member 450 may increase frictional forces among the main guide tube 200, the auxiliary guide tube 300, and the stent 50 and may prevent the stent 50 from moving, along with the support member 410.

Also, the one end and other end of the stent 50 may be fixed to the main guide tube 200 and the auxiliary guide tube 300, respectively, by the fixing wire 500 such that the stent 50 may be prevented from extending.

Here, as described above, the fixing wire 500 may be inserted into the sheath 100 through the protrusion portion 160 of the sheath handle 150 and may move to the front end of the sheath 100 to couple the one end and other end of the stent 50 to the main guide tube 200 and the auxiliary guide tube 300.

The stent 50 may be moved to the installation position while the main guide tube 200 is bound to the auxiliary guide tube 300 by the fixing wire 500, and then may be unfolded and installed at the installation position when the fixing wire 500 is disengaged.

Here, the fixing wire 500 may be formed to allow a knot which fixes the stent 50 to come disengaged as one end exposed from the sheath handle 150 is pulled.

The stent insertion apparatus according to one embodiment of the present invention has been described above with reference to the drawings. Next, a process of installing the stent 50 using the stent insertion apparatus according to one embodiment of the present invention will be described with reference to FIGS. 6 to 10.

First, referring to FIG. 6, the sheath 100 may be inserted into a human body and moved to an installation position and may move to a position at which the bifurcating bile ducts 40 bifurcate from the bile duct 30.

Here, while the sheath 100 is being inserted into the human body, the second guide wire 360 may also be inserted into the human body along with the sheath 100.

Upon reaching positions of the bifurcating bile ducts 40, the first guide wire 260 and the second guide wire 360 discharged from the one ends of the main guide tube 200 and the auxiliary guide tube 300 are adjusted to be located in bifurcating bile ducts 40 on both sides.

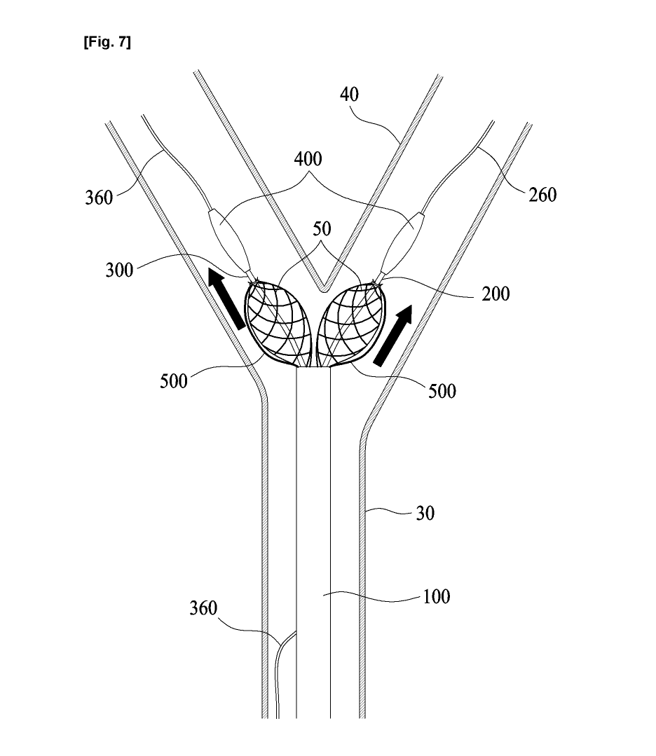

Subsequently, the guide tube handle 250 which has been described with reference to FIGS. 1 and 2 is pushed toward the sheath handle 150 such that the main guide tube 200 may be discharged from the sheath 100. That is, as shown in FIG. 7, the main guide tube 200 may be discharged from the sheath 100 and may be inserted into the bifurcating bile duct 40 along the first guide wire 260 located in the bifurcating bile duct 40.

Here, the auxiliary guide tube 300 may move as the main guide tube 200 moves and may be inserted into another bifurcating bile duct 40 adjacent to the bifurcating bile duct 40, into which the main guide tube 200 is inserted, along the second guide wire 360.

Here, the main guide tube 200 and the auxiliary guide tube 300 move from the bifurcating bile ducts 40 to the installation position of the stent 50 and then stop moving.

In this process, as the main guide tube 200 and the auxiliary guide tube 300 are discharged outward from the sheath 100, the one end and other end of the stent 50 may be inserted into the bifurcating bile ducts 40 on both sides along the main guide tube 200 and the auxiliary guide tube 300 while being bound to the main guide tube 200 and the auxiliary guide tube 300 by the fixing wire 500.

Subsequently, the main guide tube 200 and the auxiliary guide tube 300 may pull the sheath 100 to the rear end while being stopped.

As the sheath 100 moves to the rear end, the rear end of the stent 50 accommodated in the sheath 100 may be discharged outward from the sheath 100 and may expand toward an inner surface of the bile duct 30 while being discharged from the sheath 100.

Accordingly, when the sheath 100 moves to a rear end of the support member 410, the stent 50 may completely extend and may be installed in the bile duct 30.

When the rear end of the stent 50 is installed in the bile duct 30, the fixing wire 500 becomes disengaged such that the one end and other end of the stent 50 may be installed in the bifurcating bile ducts 40.

That is, when the fixing wire 500 is pulled, the one end and other end of the stent 50 coupled to the main guide tube 200 and the auxiliary guide tube 300 become disengaged and extend to be installed in the bifurcating bile ducts 40.

Accordingly, the stent 50 may be installed in a Y shape in the bifurcating bile ducts 40 and the bile duct 30.

When the stent 50 is completely installed, the main guide tube 200 may be pulled to the rear end and may be removed from the installation position of the stent 50 along with the sheath 100.

As described above, the stent insertion apparatus according to one embodiment of the present invention may be formed to install the bifurcating stent 50 at a coupling part between the bile duct 30 and the bifurcating bile ducts 40 and may be easily inserted into a human body due to the sheath 100 having a small diameter.

According to one embodiment of the present invention, a stent insertion apparatus provides effects as follows.

First, a main guide tube and an auxiliary guide tube to which bifurcating parts of a stent are coupled, respectively, are included such that the stent having a bifurcating shape can be easily installed in a bile duct.

Second, since a diameter of a sheath can be prevented from increasing even though the main guide tube and the auxiliary guide tube are included, the sheath can be easily inserted into a human body.

Effects of the present invention are not limited to the stated effects, and other unstated effects can be clearly understood by those skilled in the art from the following claims.

The embodiment of the present invention has been described above, and it will be appreciated by one of ordinary skill in the art that the present invention can be embodied in other particular forms in addition to the above-described embodiment without departing from the scope of the present invention. Therefore, the above-described embodiment should be considered not to be limitative but exemplary, and thus the present invention is not limited to the above description and may be changed within the scope of the attached claims and equivalents thereof.

What is claimed is:

1. A stent insertion apparatus comprising:
a sheath formed to have a hollow shape and in which a stent is accommodated;
a main guide tube movably inserted into the sheath and to which one end of the stent is coupled; and
an auxiliary guide tube coupled to the main guide tube in a bifurcated manner and to which the other end of the stent, which branches from the one end of the stent, is coupled.

2. The stent insertion apparatus of claim 1, comprising:
a first guide wire movably inserted into the main guide tube to guide movement of the main guide tube; and
a second guide wire inserted into the sheath through a through hole formed in an outer surface of the sheath and inserted into the auxiliary guide tube to guide movement of the auxiliary guide tube.

3. The stent insertion apparatus of claim 2, comprising olive tips respectively provided at front ends of the main guide tube and the auxiliary guide tube and formed to have a hollow shape to allow the first guide wire and the second guide wire to be inserted thereinto.

4. The stent insertion apparatus of claim 1, further comprising a support member into which the main guide tube is inserted, to which the auxiliary guide tube is coupled at an outside thereof, and which is provided at a rear end of the stent to prevent the stent from moving.

5. The stent insertion apparatus of claim 4, wherein a marker is provided at a front end of the support member to allow a position of the stent to be confirmed.

6. The stent insertion apparatus of claim 1, comprising a holding member formed to protrude from each of circumferences of the main guide tube and the auxiliary guide tube to prevent the stent from moving.

7. The stent insertion apparatus of claim 1, comprising fixing members which fix the one end and other end of the stent to the main guide tube and the auxiliary guide tube, respectively, to prevent the stent from extending.

8. The stent insertion apparatus of claim 7, comprising:
a guide tube handle provided at one end of the main guide tube to communicate with the main guide tube; and
a sheath handle coupled to and communicate with the sheath to allow the main guide tube to be inserted thereinto.
